(19) United States
(12) Patent Application Publication
(10) Pub. No.: US 2016/0375439 A1
(43) Pub. Date: Dec. 29, 2016

(54) Apparatus and Methods for Single-Particle Isolation and Single-Particle Measurement

(71) Applicants: Paul Chi Hang Li, Coquitlam (CA); Avid Khamenehfar, Vancouver (CA)

(72) Inventors: Paul Chi Hang Li, Coquitlam (CA); Avid Khamenehfar, Vancouver (CA)

(21) Appl. No.: 15/130,930

(22) Filed: Apr. 16, 2016

Related U.S. Application Data
(60) Provisional application No. 62/151,391, filed on Apr. 22, 2015.

(51) Int. Cl. B01L 3/00; G01N 15/14; G01N 33/50

(52) U.S. Cl. CPC B01L 3/502753; G01N 33/5011; G01N 15/1436; G01N 15/1484; B01L 2300/087; B01L 2400/0424; B01L 2300/0681; B01L 2200/0668; G01N 2015/0065

(57) ABSTRACT

Particles, such as cells, are isolated for conducting single-particle measurement. Isolation of rare cells, such as circulating tumor cells (CTCs), from blood is technically challenging because they are small in numbers. An integrated microfluidic biochip, dubbed as CTC chip, was designed and fabricated for conducting tumor cell isolation. As CTCs are usually multidrug resistance (MDR), the effect of MDR inhibitors on chemotherapeutic drug accumulation in the isolated single tumor cell is measured. In this invention, label-free isolation of the rare tumor cells was conducted based on cell size difference. The major advantages of the CTC chip are the ability of fast cell isolation, followed by multiple rounds of single-cell measurement, suggesting a potential assay for detecting the drug responses based on the liquid biopsy of cancer patients.

### CROSS-REFERENCE TO RELATED APPLICATION

This application claims priority to U.S. Provisional Application No. 62/151,391, filed Apr. 22, 2015. The content of the priority application is incorporated by reference in its entirety.

### TECHNICAL FIELD

The present invention relates to apparatus and methods for isolation of a single particle and measurement of the isolated single particle. An example of the single particle is a single biological cell.

### BACKGROUND

Circulating tumor cells (CTCs) were first described in 1869 by Thomas Ashworth who observed small numbers of cells in patient blood that resembled cells of the primary tumor.¹ These cells may constitute the seeds for subsequent metastasis in different organs.^{1, 2, 3} Although the nature of CTCs is not fully understood, what is widely accepted is that they have drug resistance,^{4, 5} especially multidrug resistance (MDR) due to the expression of ATP-binding cassette (ABC) transporters.^{6,7} These transporters include P-glycoprotein (P-gp or ABCB1), multidrug resistant proteins (MRP or ABCC1) and breast cancer resistant protein (BCRP or ABCG2), which cause active transport of chemotherapeutic drugs (e.g., daunorubicin or paclitaxel) out of the cancer cell. This transport is termed drug efflux, which ultimately reduces the effectiveness of chemotherapy.^{8,9} Administration of MDR inhibitors that block drug efflux mediated by MDR transporters in combination with chemotherapeutic drugs that kill the tumor cells have been explored as a potential treatment strategy.¹⁰

Isolation of CTCs can be useful for personalized cancer chemotherapy because CTCs can be clinically important to provide predictive information for the adjustment of the therapeutic scheme.¹¹ A vision is that drug accumulation measured on CTCs can provide reliable information for patients undergoing chemotherapy. However, a key limitation in the capture of CTCs is their extreme rarity in blood, i.e. the number of CTCs can be as low as ˜1-100 in 1 mL blood including 10⁹ erythrocytes or red blood cells (RBCs) and 10⁷ leukocytes or white blood cells (WBCs).¹²⁻¹⁵

Currently, CellSearch™ is a CTC-based system to provide prognostic information for metastatic breast, prostate, and colon cancers.¹⁶⁻¹⁹ In this FDA-approved system, CTCs are immunomagnetically captured from 7.5 mL of blood using magnetic beads conjugated to an antibody against the epithelial cell adhesion molecule (or EpCAM) on the cells, and then fluorescently stained with labeled antibodies against epithelial cell-specific markers.²⁰ While the system allows the CTCs to be remunerated for cancer prognosis, further cellular analysis cannot be applied because the CTCs are bound and fixed.²¹

Recently, worldwide efforts have been made to develop efficient and reliable CTC isolation techniques, such as flow cytometry.^{22, 23, 24, 25} Furthermore, a wide variety of microfluidic techniques have been reported to isolate CTCs, and the isolation methods are based on immunoaffinity,²⁶⁻³² and immunomagnetic separation.³³⁻³⁵ The immuno-based methods depend on the use of an immunological label that recognizes the EpCAM biomarker in order to identify the presence of CTCs. Therefore, there are some limitations in this immuno-label method as some CTCs, particularly those of metastatic nature, might undergo epithelial-to-mesenchymal transition (EMT), thereby losing the EpCAM marker, and potentially go undetected.^{21, 36}

On the other hand, there are some microfluidic methods that are label-free for CTC isolation, namely dielectrophoresis-based separation,³⁷⁻⁴⁰ density-based separation,^{35,41} and size-based separation.^{42,43} The last method is successful in isolating rare cells because most epithelial cells such as CTCs have sizes in order of 15-25 μm, which are larger than red blood cells (6-8 μm) and white blood cells (8-14 μm).⁴⁴

Based on the foregoing, a microfluidic biochip (CTC chip) was designed and fabricated to isolate prostate cancer (PCa) cells among whole blood cells without the use an antibody label (i.e., EpCAM antibody), followed by multiple rounds of single-cell measurements. In this approach, the human prostate cancer cells (as a model of CTC) were mixed with mouse blood cells. After removal of red blood cells and plasma, the buffy coat (white blood cells) mixed with tumor cells were introduced into the CTC chip. Since the captured cancer cell had not been subjected to any immunoaffinity manipulations (i.e., antibody), the captured cell can be used for single-cell measurements such as the drug accumulation assay.⁴⁵⁻⁴⁷ This is an established assay used to measure the real-time effect of MDR inhibitors on accumulation of chemotherapeutic drugs (e.g., daunorubicin and fluorescently-labeled paclitaxel) in the same single prostate cancer cell.

### BRIEF SUMMARY OF THE INVENTION

One aspect of the invention provides an apparatus and a method for single-particle isolation and single-particle measurement. An example embodiment of the apparatus comprises a microfluidic device that includes two fluid chambers in fluid communication with each other. The first chamber is used for separating target particles from non-target particles in a sample that comprises both the target particles and the non-target particles. The second chamber that comprises a particle retention region for retaining a single target particle is used for single-particle measurement of the target particle.

An example of the target particle is a cancer cell, and an example of the non-target particle is a white blood cell.

In some embodiments, the microfluidic device is made of glass, quartz, plastic, polymer or some other suitable transparent material.

The first fluid chamber of the microfluidic device is an elongate fluid chamber having a first end and a second end. The microfluidic device comprises a first reservoir which is in fluid communication with the first end of the first fluid chamber, the first reservoir serving as a sample inlet reservoir. In some embodiments, the sample is a blood specimen from a subject, and the particles are biological cells.

The first fluid chamber of the microfluidic device comprises a particle selection means. In one embodiment, the particle selection means is a cross-flow microfilter which comprises side openings along the side of the chamber. The side openings have a size that allows the non-target particles to pass through, but does not allow the target particles to pass through. The side openings are generally perpendicular to a longitudinal direction of the first fluid chamber.

The microfluidic device also comprises a second reservoir which is in fluid communication with the side openings of the first fluid chamber, the second reservoir serving as a non-target particle collection reservoir.

The sample flows in a direction from the first end of the first fluid chamber to the second end of the first fluid chamber, the non-target particles move toward the side openings whereas the target particles move in a middle portion of the first fluid chamber toward the second end.

The first fluid chamber separates the target particles from the non-target particles based on size difference between the target particles and the non-target particles. In some embodiments, the target particles are greater in size than the non-target particles. For example, the target particles have a size greater than 15 μm, and the non-target particles have a size in the order of 6 to 14 μm. In some embodiments, the target particles have greater inertia than the non-target particles.

In some embodiments, the target particles are cancer cells. For examples, the target particles are circulating tumor cells (CTC). In some embodiments, the non-target particles comprise non-cancer cells. For example, the non-cancers are red blood cells or white blood cells or both. The amount of the target particles is less than that of the non-target particles. For example, the amount of the target particles is 1 to 1,000,000, or 1 to 1,000, or 1 to 100, in 1 mL of the blood sample.

In some embodiments, the target particles exiting the second end of the first fluid chamber are live cells. The live cells are free of labels. For example, the live cells are free of antibody labels.

The target particles exiting the second end of the first fluid chamber will reach a second fluid chamber. The microfluidic device comprises a third reservoir which is in fluid communication with a channel connecting the first fluid chamber and the second fluid chamber, for moving one or more target particles from the first fluid chamber to the second fluid chamber.

In some embodiments, the second fluid chamber comprises a plurality of electrodes extending into the particle retention region for applying a dielectrophoretic (DEP) force to one of the target particles to control the location of target particle within the particle retention region.

In some embodiments, the second fluid chamber of the microfluidic device comprises a plurality of particle retention regions, each particle retention region for retaining at least one particle therein.

A microfluidic flow system is also used for controllably moving the target particle within the second fluid chamber by fluidic force in addition to DEP force.

The microfluidic device comprises a fourth reservoir which is in fluid communication with the second fluid chamber for delivering a reagent to the particle retention region. The microfluidic device also comprises a fifth reservoir which is in fluid communication with the second fluid chamber for collecting waste from the second fluid chamber.

An example embodiment of the method is introducing the sample comprising both the target particles and the non-target particles into the first fluid chamber, separating the target particles from the non-target particles in the first fluid chamber, flowing the target particles from the first fluid chamber to the second fluid chamber, and controlling the location of at least one of the target particles in the particle retention region in the second fluid chamber to maintain the target particle in a desired location.

The separating step comprises isolating the target particles from the non-target particles based on their size difference. The non-target particles are caused to pass through side openings of the first fluid chamber, and the target particles are caused to flow through the first fluid chamber and then into the second fluid chamber.

To conduct a single-particle measurement, a reagent is exposed to a target particle in the particle retention region. The target particle in the particle retention region is a cancer cell. The method comprises measuring one or more physical, chemical and/or biological characteristics of the cancer cell in the particle retention region after exposure of the cancer cell to a reagent, and the method further comprises measuring the region surrounding the cell for background correction purpose.

In some embodiments, the reagent comprises a chemotherapeutic drug. In other embodiments, the reagent comprises a chemotherapeutic drug and a multidrug resistance (MDR) inhibitor.

In some embodiments, the method will generate personalized chemotherapy options for the cancer patient based on the measurements of the circulating tumor cell (CTC) in the microfluidic device.

### BRIEF DESCRIPTION OF DRAWINGS

In drawings which show non-limiting embodiments of the invention:

FIG. 1: The CTC chip. (a) Image of the microchip with channels filled with a blue food dye and electrodes connected to electrical wires. (b) Layout of the microfluidic device showed Reservoir D used for drug delivery; whereas, Reservoirs A & E served as the cell inlet and waste, respectively. Blood cells were collected in Reservoir B. Reservoir C was used to protect electrodes located in Chamber 2 during on-chip HF etching of glass, and to induce a liquid flow to move target cells from Chamber 1 to Chamber 2. (c, d) Close-up images of the cross-flow microfilter in Chamber 1 in order to isolate prostate cancer cells from blood cells based on size difference. (e, f) Close-up images of the cell retention region in Chamber 2 and three DEP electrodes located inside the cell retention region to capture the prostate cancer cell isolated from a large population of blood cells, followed by drug accumulation measurement. Scale bar: 100 μm. By applying voltage between electrodes 1 and 2 for 5 min, the cell moved to the edge of electrode 1.

FIG. 2: Creation of sideward openings in the cross-flow microfilter in Chamber 1 inside a bonded glass chip (see FIG. 1c). Images (top view) showing the microfilter: (a, b) before etching, (c, d) after etching. Scale bar: 50 μm.

FIG. 3: Isolation of white blood cells through a Ficoll gradient. (a) Different layers of blood cells (from top layer to bottom: plasma+platelets, buffy coat, Ficoll solution, and red blood cells) are shown after centrifugation. The buffy coat layer contains white blood cells (i.e., lymphocytes, monocytes) and residual red blood cells. (b) Pellets included white blood cells and some red blood cells. The red blood cells were removed with a pipette carefully. Thereafter, the remaining pellet including white blood cells were resuspended in phosphate buffered saline (PBS) and centrifuged. (c) Cell pellet, mostly included white blood cells, remained after removing supernatant.

FIG. 4: Separation principle of prostate cancer cell (22Rv1) among other blood cells (WBCs+RBCs) in a cross-flow microfilter. (a) The blood cells injected alone moved toward each of sideward openings; splitting into two positions at right and left sides. (b) After injecting 22Rv1 cells alone, these bigger cells kept their straight pathway to reach Chamber 2. (c) The WBCs+RBCs went to the waste Reservoir B, and the 22Rv1 cell moved to Chamber 2 for the subsequent drug accumulation experiment. It took ˜2 s, 4 s and 8 s for each RBC, WBC and 22Rv1 cell to pass through Chamber 1, respectively. The black arrows, white arrows and gray dashed-line arrows represent the movements of 22Rv1, WBC and RBC, respectively. Scale bar: 200 μm.

FIG. 5: Steps of the isolation of cancer cells from blood cells. After isolating white blood cells through a Ficoll gradient, the cell pellet was resuspended in an appropriate medium. Images showing the morphology and size of red blood cells only (a), white and red blood cells after first PBS washing (b) and after second PBS washing (c), and mixing of prostate cancer (PCa) cell with white blood cells (1:4000 ratio) (d). The first 3 images were taken on a slide; whereas the last image was taken in the inlet Reservoir A of the CTC chip. Scale bar: 50 μm.

FIG. 6: Isolation of prostate cancer (PCa) cells from blood cells. (a) CTC chip image (schematic shown in FIG. 1b) to indicate different CTC chip regions. (b1-9) Images to show isolation of the PCa cell among WBCs and RBCs, followed by capturing the cell inside the cell retention structure in chamber 2. (c) Close-up image from b2 to show direction of prostate cancer cell movement. The black arrows, white arrows and gray dashed-line arrows represent the movements of 22Rv1, WBC and RBC, respectively. (d) Close-up image from b9 to show the capture of the PCa cell near the DEP electrode for drug accumulation experiment. Scale bar: 50 μm.

FIG. 7: Drug accumulation measured on a single PCa cell. (a) The cellular fluorescent intensity due to accumulation of 35 mM daunorubicin (DNR) was measured in real time. Fₜ is total fluorescent intensity; Fᵢ is intracellular fluorescent intensity; Fₑ is extracellular fluorescent intensity. The chip was shuttled to move the cell (b) into and (c) out of the detection window (red box) to measure the signal (Fₜ) and background (F_{b}), respectively. An AC electric field of 11.5 V at 3 MHz was applied to capture the cell close to the top DEP electrode before running the experiment. The electric field was turned off during the entire drug accumulation experiment.

FIG. 8: Optimization of the concentrations of DNR and Oregon Green-labeled paclitaxel (OG-PTX) for drug accumulation measurements. (a, b)The accumulation signal (535±92 and 510±88 cps) obtained from 35 μM of DNR and 3 μM of OG-PTX showed achievement of reasonable drug accumulation signals on 22Rv1 prostate cancer cells. (c, d) Signal remained at a saturation level in the single 22Rv1 cells treated in multiple times (4000 s) with only 35 μM of DNR (c) and only 3 μM of OG-PTX (d).

FIG. 9: Drug accumulation in a single 22Rv1 cell in the presence of fumitremorgin C (FTC) as a MDR inhibitor. (a) Fluorescent intensity of the cell treated with 35 μM DNR measured in real time. In the top curve, the drug accumulation signal obtained after adding 40 μM of FTC was enhanced and the fold-increase was 3.5±0.3. The bottom curve shows the signal remains at a saturating level in the single cell with DNR only (negative control, with 1.3±0.2 of fold-increase). (b) The fold-increase at different time points showing enhancement of drug accumulation after adding FTC (solid line, p<0.0001), as compared to the negative control (dashed line, p>0.1). (c) The images showing the cell before, during and after experiment, followed by trypan blue treatment to confirm cell viability. Scale bar: 20 μm.

FIG. 10: Trypan blue treatment conducted in Chamber 2 of the CTC chip (0-8 s). The flow of the blue dye from Reservoir D (see FIG. 1) to reach the cell retention structure is achieved in a short time (4 s). Scale bar: 200 μm.

FIG. 11: Effective concentrations of MDR inhibitors. (a) DNR accumulation in the single 22Rv1 cell in the presence of different concentrations of FTC (10, 20, 40, 80 μM) (solid line). Similar FTC experiments were conducted by single-cell accumulation of OG-PTX (dashed line). The optimal fluorescence signal was obtained after treating the cell in the presence of 40 μM FTC. (b) DNR accumulation in the single 22Rv1 cell in the presence of cyclosporine (CsA) at different concentrations of 0.5, 2.5, 5, 10 and 20 μM (solid line). Similar CsA experiments were conducted by single-cell accumulation of OG-PTX (dotted line). The optimal fluorescence signal was obtained after treating the cell in the presence of 5 μM CsA.

FIG. 12: Anti-Pgp antibody binds to the 22Rv1 cells, but not white blood cell, after drug accumulation experiments. MDR inhibitors enhanced (a) DNR accumulation and (b) OG-PTX accumulation in captured single cells, respectively. The fluorescence signal increased after adding P-gp antibody, which means single PCa cells were stained by antibody for Pgp recognition (as a specific marker for MDR cancerous cells). (c) Drug accumulation continued to rise in WBC cell after treating the cell with 35 μM of DNR alone and no drug enhancement observed after adding DNR in the presence of CsA. The WBC was not stained by antibody as expected. The cell images were depicted before and after experiment, followed by adding trypan blue in PCa cells treated with DNR (d), OG-PTX (e) and WBC (f). Scale bar: 20 μm.

FIG. 13: Effect of FTC on DNR (a) and OG-PTX accumulation (b) in the single 22Rv1 cells. The noisy and smooth lines show the raw data after normalization and after curve fitting, respectively. The images of these cells before and after treating with 35 μM of DNR (e) and 3 μM of OG-PTX (f) in the presence of 40 μM of FTC were illustrated. Adding trypan blue indicated no cell staining; hence, the cell membrane's integrity was preserved. Scale bar: 20 μm.

FIG. 14: Enhancement of DNR accumulation in single 22Rv1 cells. (a) No significant enhancement was observed (p>0.1) in single cells (black dotted line) treated for 4000 s with 35 μM of DNR alone (1.1±0.1, 1.1±0.1, 1.2±0.1)(negative control). Significant enhancement obtained after treating the single cells with drug+40 μM of FTC (black solid line) (3.7±0.2, 3.8±0.2, 3.8±0.2 fold) (p<0.0001). Black dashed line indicates partially signal decrease for DNR after removing FTC after 2000 s (3.8±0.2, 3.2±0.2, 2.7±0.2 fold). (b) Similar experiment was conducted by treating the cell with DNR in the presence of 5 μM of CsA (gray solid line). The drug accumulation enhanced by 2.6±0.2, 2.7±0.2, 2.75±0.2 fold (p<0.0001, number of data points=180). Gray dashed line (FIG. 14b) indicates more signal decrease for DNR after removing CsA (2.3±0.2, 1.9±0.2, 1.5±0.2) compared to black dashed line (FIG. 14a) after removing FTC. (c) The line was obtained by averaging the results from 6 individual cancer cell experiments, and more fold-increase in drug accumulation was observed after adding FTC, followed by CsA and combination of FTC+CsA (3.7±0.4, 4.2±0.5, 4.9±0.6 of fold-increases).

FIG. 15: Enhancement of OG-PTX accumulation in single 22Rv1 cells. (a) No significant enhancement was observed (p>0.1) in single cells (black dotted line) treated for 4000 s with 3 μM of OG-PTX alone (1.1±0.1, 1.2±0.1, 1.2±0.1)(negative control). Significant enhancement obtained after treating the single cells with drug+40 μM of FTC (3.0±0.2, 3.1±0.2, 3.1±0.2 fold increase) (black solid line). Black dashed line indicates partially signal decrease for OG-PTX after removing FTC (2.7±0.2, 1.65±0.2, 1.4±0.2). (b) Similar experiment was conducted by treating the cell with OG-PTX in the presence of 5 μM of CsA (gray solid line). The drug accumulation enhanced by 2.9±0.2, 2.9±0.2, 3.0±0.2 fold (p<0.0001, number of data points=180). Gray dashed line (FIG. 15b) indicates similar signal decrease for OG-PTX after removing CsA (2.7±0.2, 1.7±0.2, 1.3±0.2) compared to black dashed line (FIG. 15a) after removing FTC. (c) The line was obtained by averaging the results from 6 individual cancer cell experiments, and more fold-increase in drug accumulation was observed after adding FTC, followed by CsA and combination of FTC+CsA (2.7±0.3, 3.4±0.4, 3.7±0.5 of fold-increases).

### DETAILED DESCRIPTION

Throughout the following description, specific details are set forth in order to provide a more thorough understanding of the invention. However, the invention may be practiced without these particulars. In other instances, well-known elements have not been shown or described in detail to avoid unnecessarily obscuring the invention. Accordingly, the specification and drawings are to be regarded in an illustrative, rather than a restrictive, sense.

### Experimental Section

### Device Design

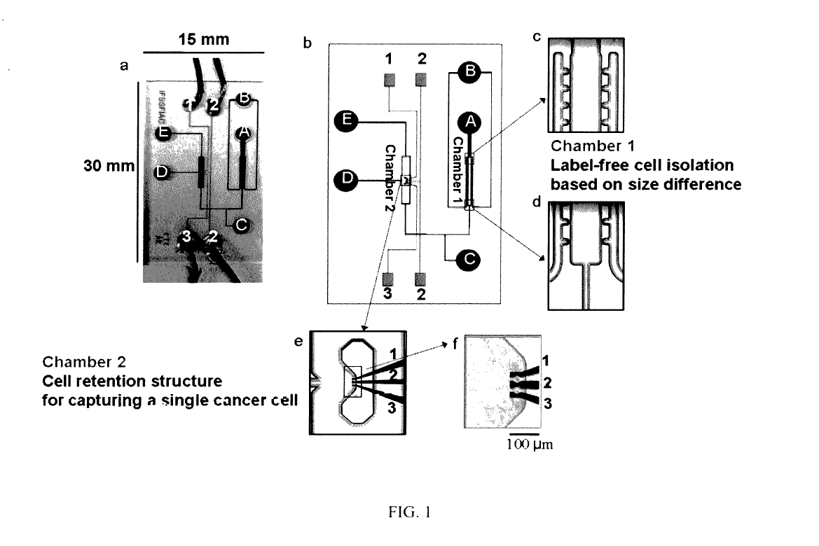

An integrated microfluidic chip, dubbed the CTC chip, was designed using the L-Edit software (Tanners). As shown in FIG. 1, the layout of the glass chip (20 mm×30 mm) consisted of two chambers: Chamber 1 containing the sideward openings, and Chamber 2 containing the cell retention structure and dielectrophoresis (DEP) electrodes. Reservoirs A, B, E served as the cell inlet, blood cell collector and waste reservoirs, respectively; Reservoir C was used to move cells from Chamber 1 to Chamber 2; Reservoir D was used for drug delivery in chamber 2. The channels and chambers in the CTC chip were 40 μm deep, while the Reservoirs were 0.6 mm deep and 2.5 mm in diameter. The CTC chip was fabricated by the standard micromachining processes at CMC Microsystems (Kingston, ON, CA). The process includes standard chip cleaning, thin film deposition, photolithography, photoresist development, HF wet etching, reservoir forming, and chip bonding, as previously described.⁴⁸

### Reagents

Daunorubicin (DNR), Oregon Green® 488-conjugated paclitaxel (OG-PTX or Flutax-2), fumitremorgin C (FTC) and cyclosporine A (CsA) were purchased from Sigma-Aldrich (St Louis, Mo.). Rowell Park Memorial Institute (RPMI) 1460 medium, trypsin-ethylenediaminetetraacetic acid (Trypsin-EDTA) (0.025%), glutamine, penicillin/streptomycin (PEN/STR) and fetal bovine serum (FBS) were obtained from Life Technologies (Grand Island, N.Y.). Hanks' balanced salt solution (HBSS) was from Invitrogen Corp (Grand Island, N.Y., USA). DNR and OG-PTX were dissolved in dimethyl sulfoxide (DMSO) (Sigma-Aldrich) to make stock solutions of 350 μM and 300 μM, respectively. Similarly, stock solution of CsA (500 μM) and FTC (1 mM) were made in DMSO. Alexa Fluor® 488-labeled anti-human P-gp monoclonal antibody was purchased from AbD Serotec (MorphoSys UK Ltd, Oxford, UK) and diluted in HBSS (1:20 ratio), and it was used to recognize human prostate cancer cell that expressed P-gp. The Ficoll-Paque PLUS solution from GE Healthcare (Pittsburgh, Pa.) was kindly provided as a gift by Dr. Naveed Gulzar at the Molecular biology and biochemistry (MBB), Simon Fraser University.

### Cell Samples

The prostate cancer (PCa) cell line, 22Rv1, obtained from ATCC, is an androgen independent, non-metastatic human cell line which naturally expresses ABCG2.^{50, 51} This PCa cell line was grown in RPMI 1460 medium supplemented with 10% FBS, 1% PEN/STR and 1% glutamine. For cell subculture, the cells were detached using trypsin-EDTA and re-seeded in fresh medium every 4 days. All cultures were maintained at 37° C. in a humidified incubator (5% CO₂ and 95% air) (NuAire). Prior to cancer cell isolation, the size of the cells was measured in order to determine the average sizes, and the cells were counted using a hemocytometer. Mouse blood cells were obtained from the Animal Care Services at Simon Fraser University after protocol approval.

### On-Chip HF Etching in Chamber 1 to Create Cross-Flow Filter

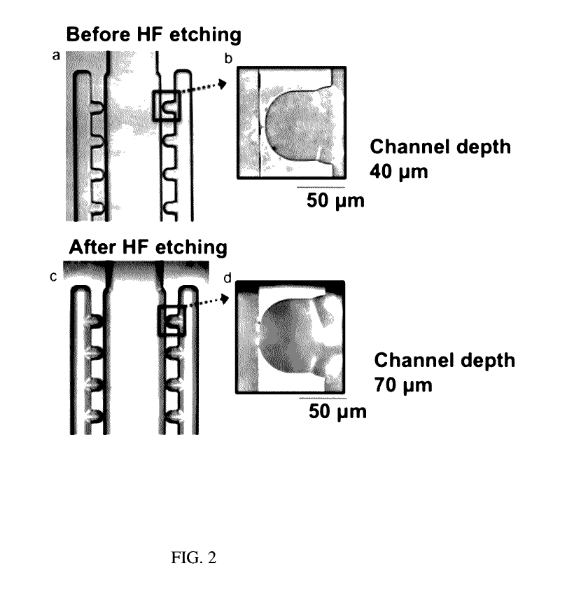

On-chip HF etching has been previously reported to enlarge a channel to create a weir structure to retain a single cardiomyocyte cell.⁴⁸ Here, on-chip HF etching was performed to create the cross flow filter in Chamber 1 in order to remove the blood cells but not the cancer cells. The spacing of the sideward openings was small enough to allow passing of the smaller blood cells (6-14 μm); whereas the larger tumor cells (15-25 μm) did not approach the sideward openings and leak through. Since a spacing smaller than 80 μm cannot be made by glass etching used to create the 40 μm-deep channels, HF etching was conducted after the glass chip was bonded. Briefly, 12% HF solution was put into Reservoir A, which was close to Chamber 1. In order to prevent HF from reaching the DEP electrodes located in Chamber 2 and destroying them, water was introduced from Reservoir C and the water flow allowed HF etching to be localized in Chamber 1. After 90 min, HF was removed from the chip to stop etching, and then the sideward openings were examined under the microscope (FIG. 2).

### Buffy Coat Preparation Using a Ficoll-Paque Gradient

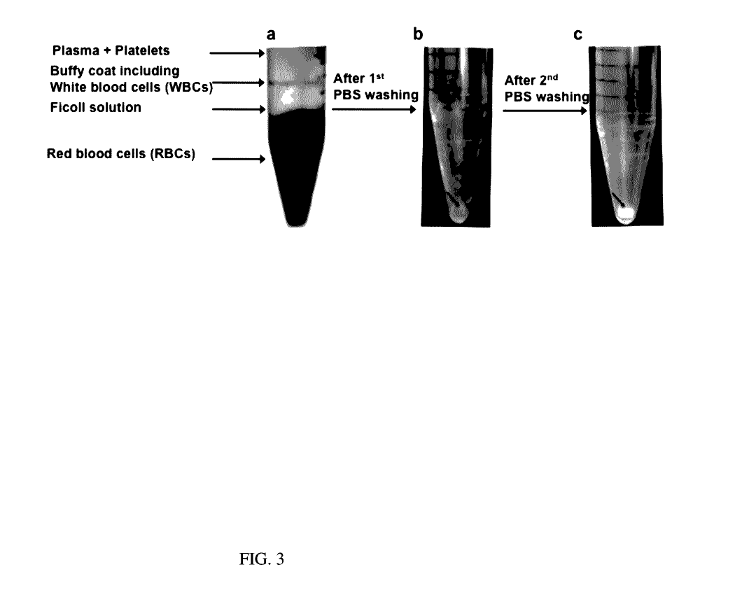

A 2-mL sample of mouse blood was collected in a tube containing the heparin anticoagulant, and white blood cells were isolated by centrifugation using a Ficoll gradient, according to the manufacturer's protocol (GE Healthcare, Pittsburgh, Pa.). Briefly, a diluted suspension of blood was layered over 3 mL of Ficoll-Paque solution in a 15-mL conical tube and centrifuged at 400×g for 40 min at 20° C. The top layer including plasma and platelets was removed; the buffy coat that consisted of the mononuclear white blood cells (FIG. 3a) was then transferred to a new 15 mL conical tube, re-suspended with PBS. After the tube was centrifuged at 500×g for 15 min., the cell pellet was collected. Since it was pink in color, it contained residual red blood cells. A micropipette tip was dipped into the cell pellet to remove the red blood cells very gently. Thereafter, the cells were re-suspended in PBS (FIG. 3b), and centrifuged at 500×g for 15 min. The cell pellet was washed one more time with PBS by spinning at 600×g for 8 min (FIG. 3c). Cells were then re-suspended in RPMI 1460 medium (supplemented with 10% FBS and 1% PEN/STR and 1% glutamine) for cancer cell isolation.

### Isolation of Individual Prostate Cancer Cells

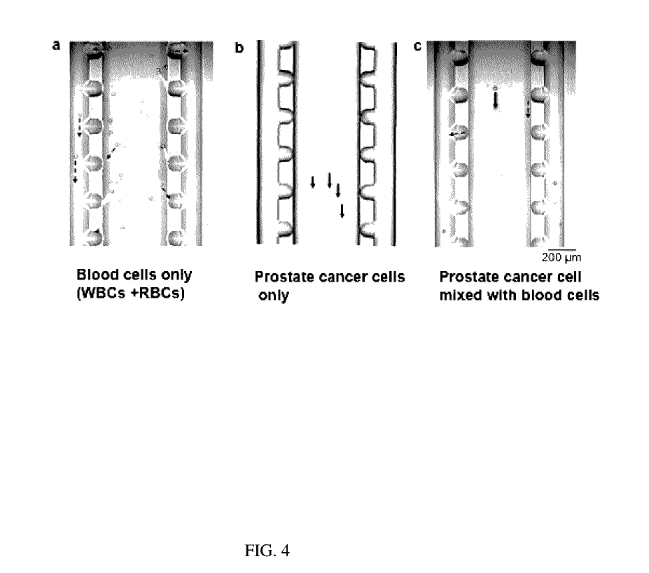

The prostate cancer cell (22Rv1) was isolated using the cross-flow microfilter in Chamber 1. FIG. 4 describes the separation principle of the 22Rv1 cell among blood cells (i.e., WBCs+RBCs). Once the mixed cell sample (22Rv1 cells +blood cells in a ratio of 1:4000) entered the wide chamber region (Chamber 1), according to inertia, the heavier and larger 22Rv1 cell continued its straight pathway by the primary flow (FIG. 4c), while smaller and lighter cells followed the sideward flow (FIG. 4c). Comparison of FIG. 4a (blood cells only) and FIG. 4b (22Rv1 cells only) confirmed that smaller cells moved toward the sideward openings, while the larger 22Rv1 cell continued the straight trajectory in the middle along Chamber 1. This result suggested that the inertia of the larger 22Rv1 cell prevented it from any sideward movement regardless of the presence of blood cells.

Prior to the CTC capture experiment, the channels and chambers were filled with culture medium (RPMI 1460 supplemented with 10% FBS) for 15 min. A cell sample containing a mix of 22Rv1 cells and blood cells (in a ratio of 1:4000) was injected into the CTC chip from the inlet Reservoir A. As soon as the 22Rv1 cells were observed in Chamber 1 and then they had moved on, they were guided toward Chamber 2 by manipulating the liquid flow using Reservoirs A, C and E. For instance, with the liquid level at Reservoir A high and those at Reservoirs C and E low, the 22Rv1 cells would leave Chamber 1. As soon as the cells were near Reservoir C, the liquid flow from it was increased to push the cell further toward the cell retention structure in Chamber 2.

### Dielectrophoresis Electrodes to Trap Single Cancer Cells in Chamber 2

The term dielectrophoresis (DEP) was first introduced by Herbert Pohl in the 1950's to describe the behaviour of particles in non-uniform electrical fields.⁵² DEP force can be created in a non-uniform electric field to move particles.⁵³⁻⁵⁵ The DEP forces depend on factors such as cell membrane and cytoplasm electrical properties as well as cell size.⁵⁶ When the DEP force and drag force that act on the cell reached equilibrium, the cell could be kept stationary. Based on this concept, DEP electrodes were used for single-cell trapping in fluorescent measurements. The proper frequency and magnitude of the alternating voltage have been optimized to retain the cell, but not damage it by high voltage.⁵⁷ Therefore, 11.5 V (3 MHz) was applied between Electrodes 1 and 2 to keep the cell stationary for experiments. The DEP force was turned off at ˜5 min after trapping the single cancer cell.

### On-Chip Drug Accumulation Measurement on Isolated Single Prostate Cancer Cell

After the cell was kept stationary, the medium was introduced into Reservoir D to induce a liquid flow to make sure the cell was stationary before running the drug accumulation experiment. An optical detection system was employed for simultaneous fluorescence measurement and bright-field imaging.^{47,58}

The procedure for drug accumulation measurement has previously been reported.⁴⁵⁻⁴⁷ Briefly, the anti-cancer drug (i.e., DNR or OG-PTX) was introduced via Reservoir D and drug accumulation of the anti-cancer drug was measured in the single cell. In the next step, the MDR inhibitor (i.e., CsA and/or FTC) was introduced via Reservoir D, and drug accumulation was measured in the same cell. Adding MDR inhibitors increased drug accumulation in the cell, and then the single-cell fluorescence intensity was enhanced. DNR was first used for drug accumulation measurement as it had inherent fluorescence (λₑₓ=470 nm; λₑₘ=585 nm). Thereafter, paclitaxel that was fluorescently labelled by Oregon Green was examined (λₑₓ=492 nm; λₑₘ=524 nm), since paclitaxel was the commonly-used anti-cancer drug for prostate cancer treatment.

### Statistical Analysis

Data were presented as the mean±SD (standard deviation). Statistical significance test was determined using the Student's t-test.

### Results of Prostate Cancer Cell Isolation Among Blood Cell

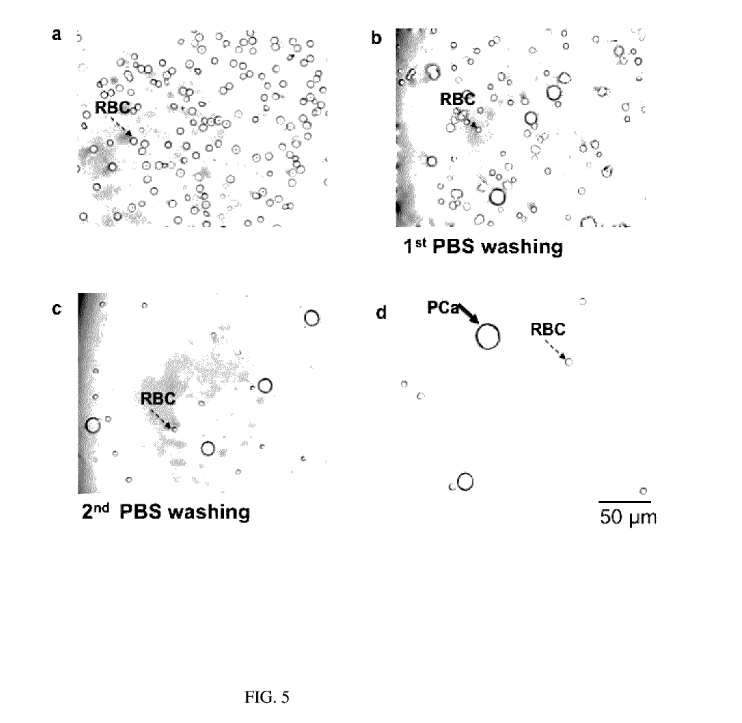

The morphology and size of human prostate tumor cells and mouse blood cells (WBCs+RBCs) were examined first. FIG. 5a shows the RBCs, and FIGS. 5b and 5c show the WBCs mixed with RBCs after one and two washes with PBS, respectively. This is a model to capture CTCs in a condition of 1 CTC in 4000 WBCs, or 250 CTCs in 10⁶ WBCs¹⁵.

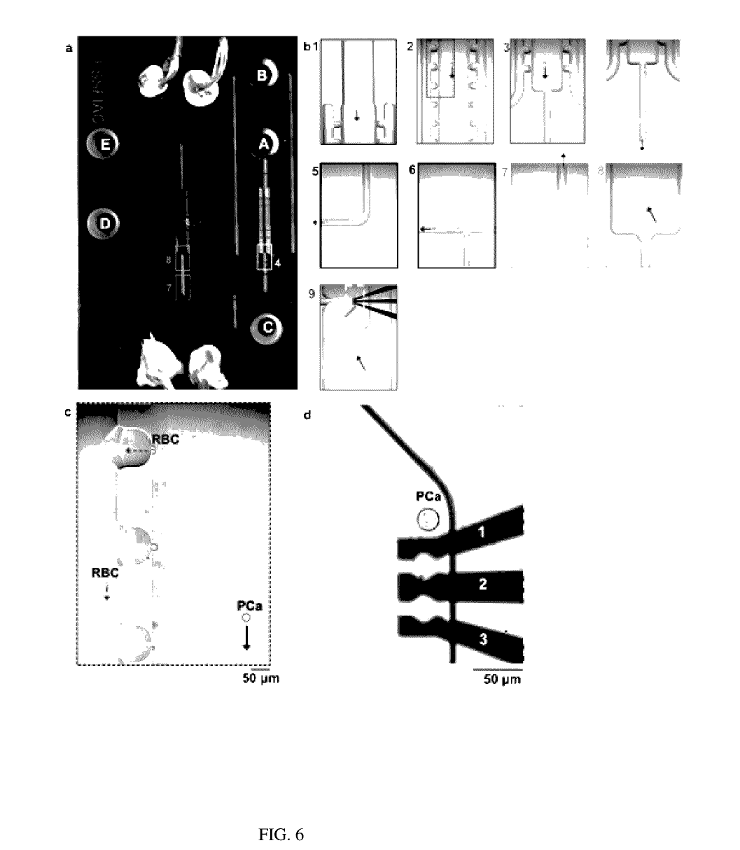

FIG. 6 shows the process for isolating the prostate cancer (PCa) cells among blood cells. Based on the size differences of the cells, blood cells were split into two directions and moved to the right and left side (FIG. 6b2-3) and collected in waste Reservoir B. The PCa cell moved in the middle of Chamber 1 (FIGS. 6b2 and 6c) since it was too big to pass through the sideward openings. Approximately 33 min after injecting the cell sample, the first PCa cell passed through the first channel, which connected Reservoir A to Chamber 1. An additional 4 min was used to move the PCa cell to be captured by the DEP electrode at the cell retention structure in Chamber 2. This is a process for cell capture without the use of an immunoaffinity label and for subsequent single-cell measurement, a process not currently feasible using conventional methods.

### Drug Accumulation Study on a Single 22Rv1 Cell

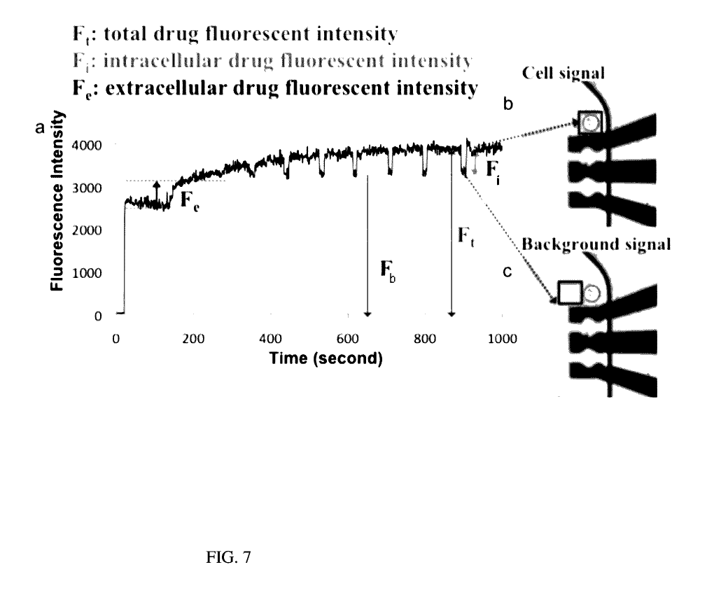

Drug accumulation was conducted in Chamber 2. In the first step, accumulation of the anti-cancer drug (i.e., DNR or OG-PTX) in the MDR single cell was measured in the absence of MDR inhibitor (FIG. 7a). The 22Rv1 cells naturally express the ABCG2 transporter, which actively transports drug molecules out of the cell.⁵⁰ As DNR is a well-known ABCG2 substrate,⁵¹ the initial accumulation of DNR without the influence of any MDR inhibitor was low. As shown in FIGS. 7b and 7c, during data collection, the chip was shuttled back and forth to allow for measurement of the cell in and out of the detection window. When the cell was inside the window, the total fluorescent intensity (Fₜ) was measured; whereas the background (F_{b}) was measured when the cell was outside the window.^{58, 59} Subtraction of the background (F_{b}) from the total fluorescent intensity (Fₜ) gave a corrected signal (Fᵢ) representing the drug concentration inside the single prostate cancer cell.^{45, 59}

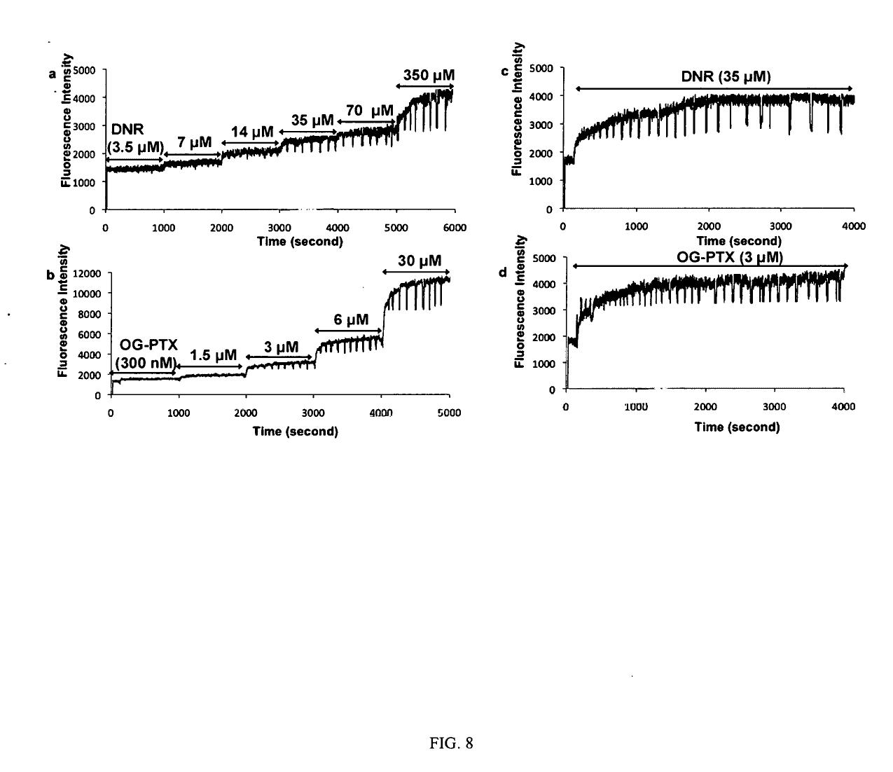

The experiment was continued using DNR at different concentrations (3.5, 7, 14, 35, 70, 350 μM) in order to determine the reasonable initial signal of drug accumulation in the cell. As shown in FIG. 8a, the corrected signal of DNR (F₁) at the above 6 concentrations were 100±37, 290±39, 260±41, 530±42, 550±47 and 1280±50 counts per second (cps), respectively. The signal obtained from DNR at 35 μM provided the optimal initial value. Similarly, other experiments were conducted on a single 22Rv1 cell by treating the cell with OG-PTX at different concentrations (0.3, 1.5, 3, 6, 30 μM). As shown in FIG. 8b, the corrected signals of the OG-PTX were 60±48, 100±52, 500±55, 1300±56, 2900±72 cps, respectively. Therefore, the optimal fluorescence signal was obtained after treating the single cell with 3 μM of OG-PTX. After the optimization experiment for drug concentration, subsequent experiments were carried out with 35 μM of DNR or 3 μM of OG-PTX. FIG. 8c shows a single 22Rv1 cell treated with 35 μM of DNR for a long period of time, showing the cell has reached a saturated fluorescent level. A similar experiment was conducted on another single 22Rv1 cell by adding 3 μM of OG-PTX, indicating saturation in the fluorescent intensity (FIG. 8d).

### Effect of FTC on DNR Accumulation

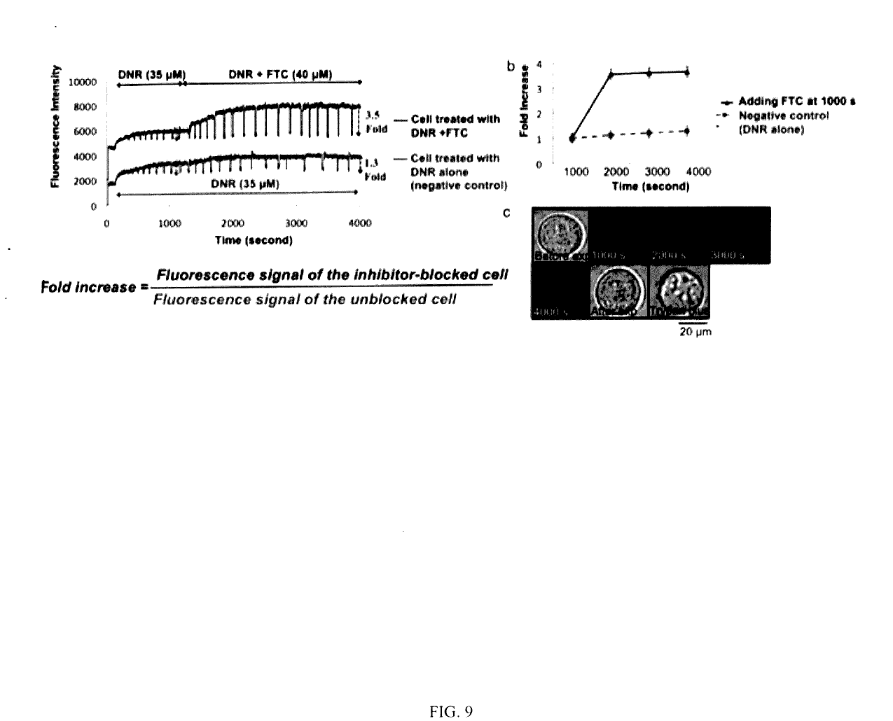

Since 22Rv1 cells highly express the ABCG2 transporter which leads to low DNR accumulation in the cells, adding fumitremorgin C (FTC, an ABCG2-specific MDR inhibitor) should increase DNR accumulation, and single-cell fluorescence should be enhanced. As shown in the top curve of FIG. 9a, a steady state or plateau of the drug accumulation signal, due to a balance of the drug uptake and efflux processes, was first obtained. With the addition of FTC, the steady state was disturbed, and the DNR accumulation increased instantly. The effectiveness of the MDR inhibitor is indicated by the fold-increase in fluorescence, which is defined as the ratio between the fluorescence signals of the inhibitor-blocked cell and that of the unblocked cell. As shown in FIG. 9a, adding FTC as a MDR inhibitor enhanced drug accumulation by 3.5±0.3 fold (top curve), as compared with the 1.3 fold obtained in the negative control (bottom curve). The fold-increases obtained at different time points before and after adding FTC were also plotted in FIG. 9b. The value of 3.5±0.3 is higher than the 2.0-fold accumulation of D-luciferin (another well-known substrate of ABCG2) obtained in 22Rv1 cells treated by FTC (25 μM), based on a time-consuming bioluminescence imaging experiment.⁶⁰

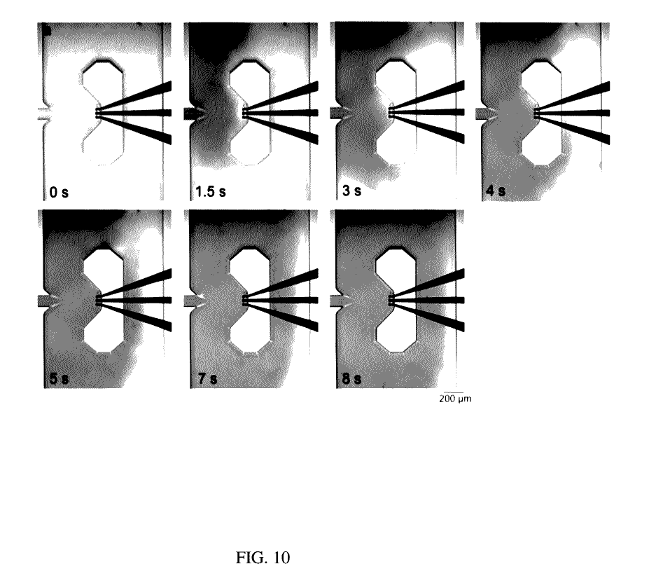

FIG. 9c shows the morphologies of this MDR cell (i.e., 22Rv1) before experimental manipulation (bright field observation), during DNR treatment (simultaneous red-light bright-field observation during fluorescence measurement), after adding DNR in the presence of FTC, and after trypan blue treatment. This dye was used to check if the cell membrane was compromised after the exposure to the various conditions, as a measure of cell viability. Therefore, the cell was viable even after DEP force was applied to capture the cell, after 35 μM of DNR was used to treat the cell, and after the xenon arc lamp was used to excite the molecules.⁴⁵ FIG. 10 shows the flow of trypan blue used to treat the cell, demonstrating how the dye quickly flows in Chamber 2 to completely reach the cell (˜4 s), consistent with the results of a recent study about fast liquid flow occurring in the cell retention structure.⁶¹

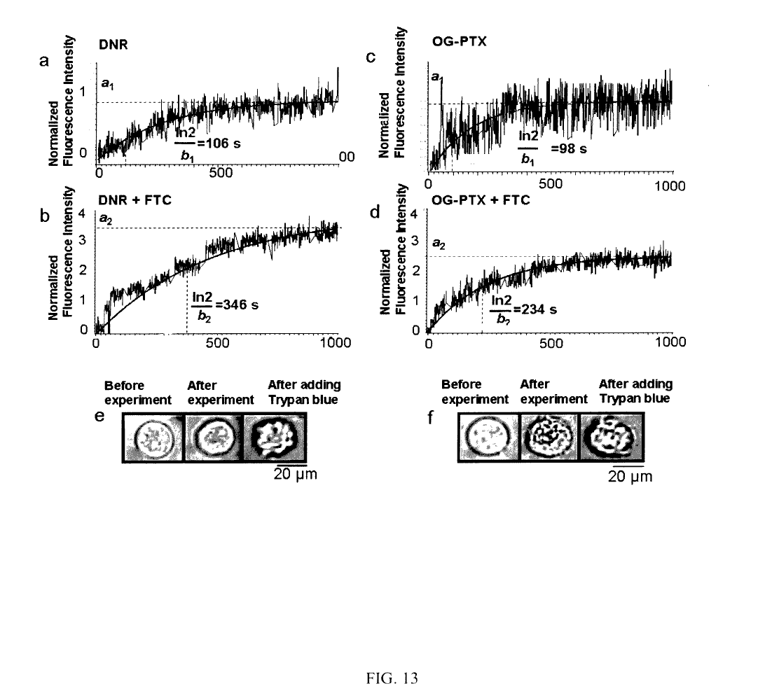

Our real-time fluorescence drug accumulation experiments also allow us to obtain the kinetics of the MDR inhibitor effects, using a previously reported mono-exponential drug uptake model.⁴⁵ The curve fitting analysis was performed on the normalized drug accumulation data, using the SAS software (see FIG. 13). Based on the curve fitting results (see [0070] to [0074]), the fold-increases for DNR and OG-PTX accumulations were determined to be 3.4±0.2 and 2.4±0.4, which were believed to be caused by the action of FTC on the ABCG2-mediated drug efflux in the 22Rv1 cell.

### Effective Concentrations of MDR Inhibitors (FTC and CsA)

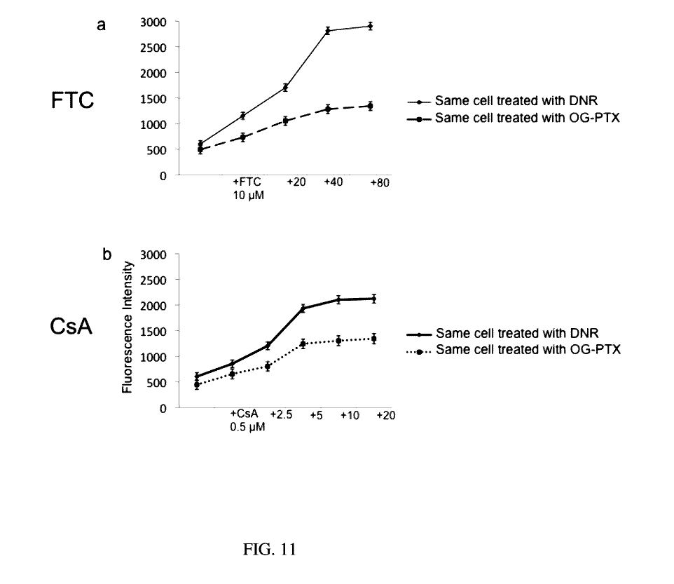

Although P-gp is weakly expressed in normal prostate,⁶² its expression increases in the tumor epithelium,⁵³ especially in androgen-independent prostate cancer.⁵⁵ For instance, P-gp (ABCB1) was detected in 35% of samples collected from non-treated prostate cancer (PCa) patients.⁵⁴ On the other hand, the ABCG2 transporter has been found in androgen-independent prostate carcinoma cells such as 22Rv1 cells,⁵⁰ and Huss et al. has reported that this transporter might mediate drug resistance in prostate cancer stem cells resistant to androgen therapy.⁶³ Therefore, drug accumulation experiments on 22Rv1 cells were evaluated using both FTC (as a well-known ABCG2 inhibitor) and CsA (as a common ABCB1 inhibitor). As shown in FIG. 11a (solid line), the fluorescence signal of the DNR was 630±77. After adding different concentrations of FTC, the signals were enhanced. Similar experiments were performed by adding different concentrations of OG-PTX (FIG. 11a: dashed line). Therefore, the optimal fluorescence signals were obtained after treating the single cells with both anti-cancer drugs in the presence of 40 μM of FTC. In a similar manner, experiments were performed on single cells to optimize the CsA concentration, and it was found to be 5 μM to maximize signal enhancement (FIG. 11b).

### Effect of Multiple MDR Inhibitors on Single Prostate Cancer Cell

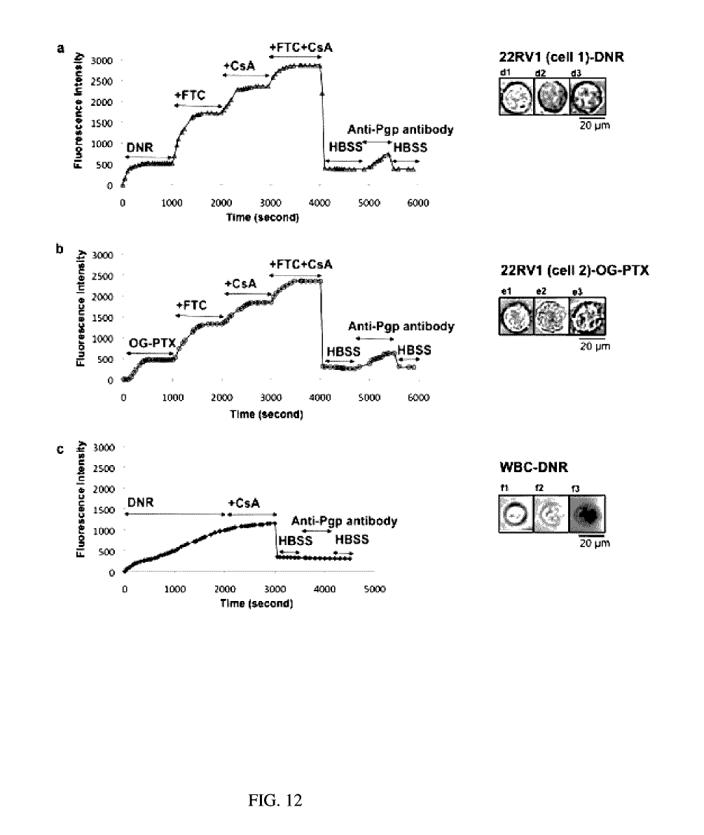

Multiple rounds of drug accumulation experiments were conducted on the single PCa cell isolated from blood cells. As a model to prove the captured single cell is indeed cancerous, Alexa Fluor® 488-labeled anti-human monoclonal P-gp antibody was introduced to detect P-gp on the 22Rv1 cell surface after drug accumulation experiments. FIG. 12a shows the DNR accumulation experiments conducted on the single 22Rv1 cell after isolating it from white blood cells (WBCs). Obvious enhancement in fluorescent intensity (at 585 nm) was observed (i.e., 3.3±0.3, 4.5±0.5, 5.4±0.5 fold-increase (p<0.0001). After washing the cell with HBSS (2×), anti-P-gp was applied and fluorescence signal (524 nm) increased from 380±62 to 710±65 cps. This result confirmed the PCa cell was cancerous since it was stained by the anti-Pgp antibody.

On the other hand, in a similar experiment conducted on the WBC (FIG. 12c), the cell was not stained by the anti-Pgp antibody, indicating it is not a PCa cell. As for the single-cell DNR accumulation, the experiment on the WBC demonstrated the behavior of a non-MDR cell, showing continuous increase in drug accumulation and no enhancement by CsA. The high DNR accumulation in the WBC also led to its staining by trypan blue (FIG. 12 f3)

FIG. 12b shows the accumulation of OG-PTX in a single 22Rv1 cell after its isolation from WBCs. Obvious enhancement in fluorescent intensity (at 585 nm) was observed (i.e., 2.8±0.3, 4.4±0.5, 4.9±0.5 fold-increase (p<0.0001). After washing the cell, anti-P-gp was applied. The fluorescence signal (524 nm) increased from 265±70 to 615±72 cps, confirming that the PCa cell was cancerous.

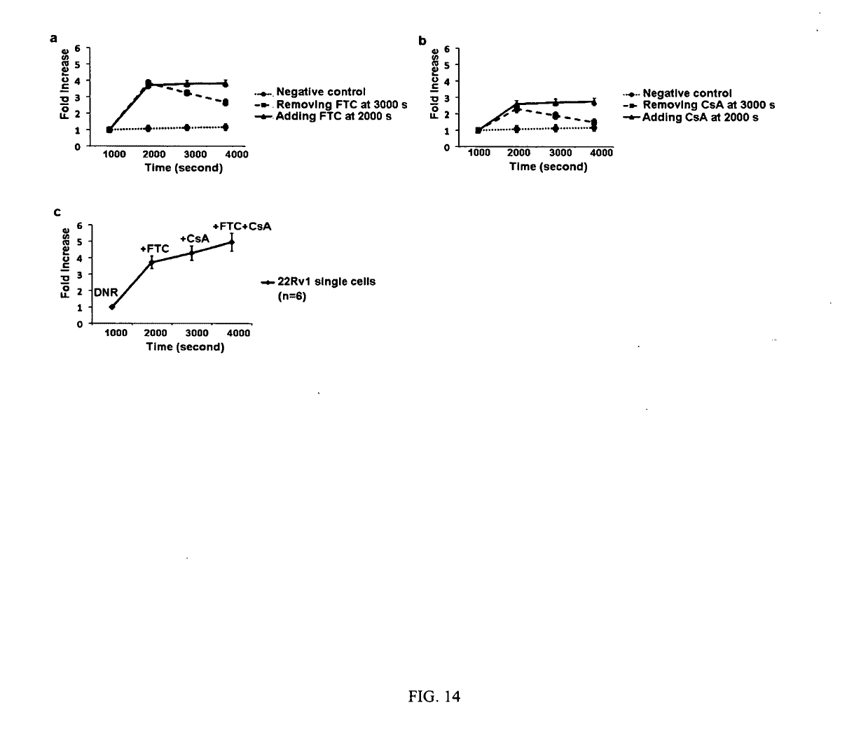

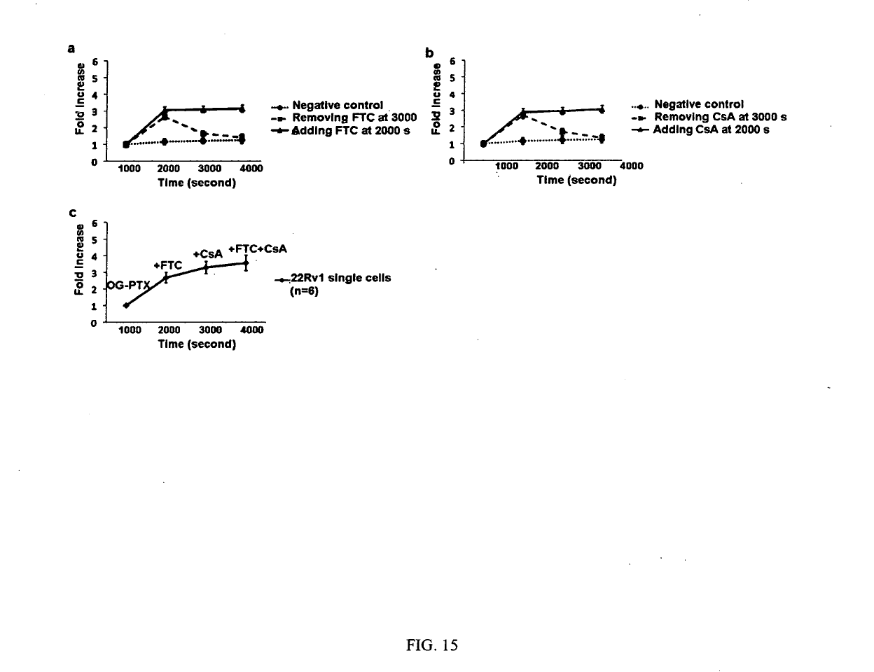

We also found the treatment of FIC+CsA after the treatment of CsA further enhanced drug accumulation in the cell due to MDR inhibition (FIGS. 12a and 12b). The CsA-mediated enhancement after plateau likely indicates additional MDR inhibition due to FTC treatment. The inhibition observed after FTC treatment could have been subsequently been lost during the CsA treatment. This loss of inhibition is confirmed in FIG. 14a (black dashed line) and FIG. 14b (gray dashed line) for the cell accumulating DNR, since the fluorescence signal intensity dropped as soon as the MDR inhibitors were removed. Similarly, for the cell accumulating OG-PTX, the fluorescence signal intensity dropped as soon as the MDR inhibitors were removed (see FIG. 15a; black dashed line and FIG. 15b: gray dashed line).

It is interesting to find that the further enhancement of drug accumulation due to treatment of FTC+CsA were only observed in the single-cell experiments, but not in averaged results when the number of experiments is insufficient. For instance, single-cell experiment revealed p values to be less than 0.001 for the enhancement after treating the same single 22Rv1 cell with DNR in combination with FTC+CsA as shown in FIG. 12a. In a similar manner, significant enhancement (p<0.001) of OG-PTX accumulation was observed when real-time measurement was conducted (number of data points=140) on the same single cell with both treatments (with CsA or with CsA+FTC) (FIG. 12b). However, the averaged results of several single-cell experiments did not result in the enhancement in a statistically significant manner (p>0.05) (FIG. 14c & FIG. 15c). With more repeated experiments, a significant enhancement in the averaged results will be observed, but the same single-cell experiment has the power to reveal the significant change when conducted real-time on the same single cell.

### Comparison of Single-Cell DNR and OG-PTX Drug Accumulation Kinetics

We previously reported that a mono-exponential drug uptake model could be applied to fit the experimental data in order to compare drug accumulation kinetics in MDR cells.⁴⁵ The following equation was used to describe the time-dependent change of single-cell DNR accumulation:

y=a(1−e^{-bx}) (1)

where x is time; y is the normalized fluorescence signal obtained by dividing the intracellular concentrations by the extracellular DNR concentrations; a is the y-axis value that will reach when the time x is sufficiently large (this value is called pre-exponential factor that is related to the cell permeability coefficient p of drug uptake and the pump rate k for drug efflux); b, which is the half time when y reaches 50% of a, is given by

$$\frac{ln 2}{b} {\cdot }^{64}$$

The curve fitting analysis was performed on the normalized fluorescence using the SAS software. The mono-exponential drug uptake model was applied to the data obtained in the absence and presence of FTC, as shown in FIG. 13a,b, which depict the normalized fluorescence of only 35 μM of DNR accumulation (step 1), and of drug accumulation enhanced by 40 μM of FTC (step 2). Because of the initially large drug concentration gradient across the cell membrane, the initial cellular accumulation rate of drugs is very fast,⁶⁵ and this fast rate is shown as a short half time of ˜106 s in FIG. 13a. When the concentration gradient became smaller, the drug uptake rate became slower; ultimately, the signal reached a plateau as the drug uptake rate was close to the drug efflux rate. In the presence of FTC, drug accumulation increased (FIG. 13b). This significant drug enhancement is believed to be caused by the action of FTC on the ABCG2 drug efflux pump. The curve fitting result is shown in Table 1. It was found that a increased from 1.0±0.3 to 3.4±0.2; b decreased from 0.010±0.003 to 0.004±0.002 s⁻¹, so the half time to reach the plateau increased from 106 s to 346 s.

In a similar manner, an experiment was conducted by treating a 22Rv1 cell by 3 μM of OG-PTX, followed by OG-PTX-loaded FTC40 of μM, as shown in FIGS. 13c and 13d. As shown in Table 2, in the presence of FTC, the a value increased from 1.0±0.1 to 2.4±0.4; whereas the b value decreased from 0.007±0.006 to 0.003±0.008, so the half-time to reach plateau increased drom 98 s to 24 s (FIG. 13c, d).

TABLE 1
Curve fitting data to show drug accumulation of DNR (35 μM)
in the same single 22Rv1 cell enhanced by FTC (40 μM).

| | |
|---|---|
| Step 1: DNR | Step 2: DNR + FTC |
| R² = 0.956 (n = 915) | R² = 0.921 (n = 900) |

| a₁ | b₁ (s⁻¹) | a₂ | b₂ (s⁻¹) |
|---|---|---|---|
| 1.0 ± 0.3 | 0.010 ± 0.003 | 3.4 ± 0.2 | 0.004 ± 0.002 |

TABLE 2
Curve fitting data to show drug accumulation of OG-PTX (3 μM) in the
same single 22Rv1 cell enhanced by FTC (40 μM).

| | |
|---|---|
| Step 1: OG-PTX | Step 2: OG-PTX + FTC |
| R² = 0.971 (n = 898) | R² = 0.948 (n = 905) |

| a₁ | b₁ (s⁻¹) | a₂ | b₂ (s⁻¹) |
|---|---|---|---|
| 1.0 ± 0.1 | 0.007 ± 0.006 | 2.4 ± 0.4 | 0.003 ± 0.008 |

### Effect of Multiple MDR Inhibitors on Single Prostate Cancer Cells

One of the key requirements for the effectiveness of chemotherapy is the sufficient delivery of chemotherapeutic drug into the cancer cell.⁶⁶⁻⁶⁹ As mentioned, drug resistance due to MDR activity is due to the function of ABC transporters that reduce intracellular drug accumulation. In that regard, we tested whether FTC and CsA modulated BCRP and P-gp function and improved the retention of DNR and OG-PTX in single PCa cells. In the single 22Rv1 cells treated with DNR in the presence of FTC (FIG. 14a: black solid line) and in the presence of CsA (FIG. 14b: gray solid line), an approximate (3.7±0.2, 3.8±0.2, 3.8±0.2) and (2.6±0.2, 2.7±0.2, 2.75±0.2) fold-increase in DNR accumulation after 1000 s of adding these MDR inhibitors was observed. Similar results were obtained after applying OG-PTX in the presence of same MDR inhibitors (FIG. 15a: black solid line & FIG. 15b: gray solid line) (3.0±0.2, 3.1±0.2, 3.1±0.2, and 2.9±0.2, 2.9±0.2, 3.0±0.2 of fold-increase, respectively). However, in the single cell treated with OG-PTX, the fluorescence signal decreased in the cells once MDR inhibitors (FTC and CsA) were removed (FIG. 15a: black dashed line and FIG. 15b: gray dashed line). On the other hand, after accumulation of DNR in the presence of MDR inhibitors, DNR was more effectively retained in MDR cells following removal of MDR inhibitors, particularly FTC from single MDR-treated cell (FIG. 14b: black dashed line).

DNR, which intercalates between the DNA bases and impairs DNA synthesis in the cell nucleus, may get aggregated to form clusters in the nucleus that are too big to pass out through the nuclear pores in a short time (˜1 hr);^{67,68} therefore, it might help DNR molecules to retain for a longer time in the cell as compared to OG-PTX. Unlike DNR, OG-PTX's site of action is the cytoplasm. This fluorescently labeled anti-cancer drug molecule functions by stabilizing tublin polymerization in the cytoplasm, ultimately it can interfere cell division.⁶⁹ Based on the current results, we believe that once DNR enters the cell, it translocates into the nucleus; therefore, it may not be as vulnerable as OG-PTX to be extruded from the cells once the MDR function is restored. More experiments were performed on DNR accumulation in single MDR cells using different inhibitors. Applying FTC, CsA and FTC+CsA produced fold-increases of 3.7±0.4, 4.2±0.5, 4.9±0.6, respectively (FIG. 14c). Additional experiments by treating single cells with OG-PTX indicated similar results (2.7±0.3, 3.4±0.4, 3.7±0.5) (FIG. 15c). It should be pointed out that ABCB1 appears to be the main PTX transporter, while DNR is the substrate of both ABCB1 and ABCG2 transporters.⁷⁰ As explained above, there was a significant signal decrease after removing the MDR inhibitors. This signal decrease due to inhibitor loss was recovered by adding more MDR inhibitors (i.e. CsA and combination of FTC+CsA). We also found the combination of FTC and CsA enhanced drug accumulation more when the cells were treated with DNR (FIG. 14c). Although the inhibition mechanism of the combination of MDR inhibitors is not entirely clear, the simultaneous administration of MDR inhibitors has been reported. For instance, verapamil & CsA (as P-gp inhibitors) and MK-571 (as a MRP1 inhibitor) has been reported to increase the intracellular levels of [3H]-azacytidine in leukemia cells.⁷¹

In this study, the applicability of a new microfluidic biochip for the label-free isolation of prostate cancer (PCa) cells as a model of CTC capture was demonstrated. This integrated chip has the capability to improve capture of single cells from blood cells as well as to preserve cell viability for subsequent drug measurement. Multiple rounds of drug accumulation experiments can then be conducted on the viable single cell to investigate the effect of MDR inhibitors on anti-cancer drug accumulation. FTC (as a well-known ABCG2 inhibitor) and CsA (as a P-gp inhibitor) are found to be effective in the enhancement of drug accumulation in the captured single PCa cells. The advantages of this integrated chip are the ability of fast isolation of PCa (<1 hr), of drug measurement (˜1 hr) and of confirming the identity of the P-gp-expressing cancerous cell. This new biochip requires a small number of cells compared to conventional methods to confirm the response to MDR inhibitors, providing a potential for CTC research and for investigating MDR effects in CTCs.

It is understood that the examples in the foregoing disclosure in no way serve to limit the scope of this invention, but rather are presented for illustrative purposes. As will be apparent to those skilled in the art in the light of the foregoing disclosure, many alterations and modifications are possible in the practice of this invention without departing from the scope thereof.

This invention has a wide range of aspects. Without limitation, the aspects include each of the following:

1. A microfluidic device comprising:
(a) a first fluid chamber for separating target particles from non-target particles in a sample that comprises both the target particles and the non-target particles,
(b) a second fluid chamber which comprises a particle retention region for retaining at least one of the target particles, and wherein the first fluid chamber is in fluid communication with the second fluid chamber.
2. The microfluidic device according to aspect 1, wherein the first fluid chamber comprises particle selection means.
3. The microfluidic device according to aspect 2, wherein the particle selection means comprises side openings along at least a side of the first fluid chamber.
4. The microfluidic device according to aspect 3, wherein the side openings of the first fluid chamber have a size that allows the non-target particles to pass through, but does not allow the target particles to pass through.
5. The microfluidic device according to any one of aspects 2 to 4, wherein the first fluid chamber is an elongate fluid chamber having a first end and a second end.
6. The microfluidic device according to aspects 3 to 4, wherein the side openings are generally perpendicular to a longitudinal direction of the first fluid chamber.
7. The microfluidic device according to aspect 5, wherein the microfluidic device comprises a first reservoir which is in fluid communication with the first end of the first fluid chamber, the first reservoir serving as a sample inlet reservoir.
8. The microfluidic device according to aspect 5, wherein the microfluidic device comprises a second reservoir which is in fluid communication with the side openings of the first fluid chamber, the second reservoir serving as a non-target particle collection reservoir.
9. The microfluidic device according to any one of aspects 5 to 8, wherein when the sample flows in a direction from the first end of the first fluid chamber to the second end of the first fluid chamber, the non-target particles move toward the side openings whereas the target particles move in a middle portion of the first fluid chamber toward the second end.
10. The microfluidic device according to any one of aspects 5 to 9, wherein the first fluid chamber separates the target particles from the non-target particles based on size difference between the target particles and the non-target particles.
11. The microfluidic device according to aspect 10, wherein the target particles are greater in size than the non-target particles.
12. The microfluidic device according to any one of aspects 5 to 11, wherein the target particles have greater inertia than the non-target particles.
13. The microfluidic device according to any one of aspects 1 to 12, wherein the sample is a blood specimen from a subject.
14. The microfluidic device according to aspect 10, wherein the target particles are cancer cells.
15. The microfluidic device according to aspect 14, wherein the target particles are circulating tumor cells (CTC).
16. The microfluidic device according to aspect 10, wherein the non-target particles comprise non-cancer cells such as red blood cells or white blood cells or both.
17. The microfluidic device according to aspect 11, wherein the target particles have a size greater than 15 μm.
18. The microfluidic device according to aspect 17, wherein the target particles have a size in the order of 15 to 25 μm.
19. The microfluidic device according to aspect 13, wherein the non-target particles have a size less than 15 μm.
20. The microfluidic device according to aspect 19, wherein the non-target particles have a size in the order of 6 to 14 μm.
21. The microfluidic device according to any one of aspects 13 to 20, wherein the amount of the target particles in the blood sample is 1 to 1,000,000 in 1 mL of the blood sample.
22. The microfluidic device according to aspect 21, wherein the amount of the target particles in the blood sample is 1 to 1, 000 in 1 mL of the blood sample.
23. The microfluidic device according to aspect 22, wherein the amount of the target particles in the blood sample is 1 to 100 in 1 mL of the blood sample.
24. The microfluidic device according to any one of aspects 11 to 23, wherein the target particles exiting the second end of the first fluid chamber are live cells.
25. The microfluidic device according to aspect 24, wherein the live cells are free of labels.
26. The microfluidic device according to aspect 25, wherein the live cells are free of antibody labels.
27. The microfluidic device according to any one of aspects 1 to 26, wherein the second fluid chamber comprises a plurality of electrodes extending into the particle retention region for applying a dielectrophoretic (DEP) force to said at least one of the target particles to control the location of said particle within said particle retention region.
28. The microfluidic device according to aspect 27, wherein the microfluidic device comprises a third reservoir which is in fluid communication with a channel connecting the first fluid chamber and the second fluid chamber, for moving one or more target particles from the first fluid chamber to the second fluid chamber.
29. The microfluidic device according to aspect 28, wherein the microfluidic device comprises a fourth reservoir which is in fluid communication with the second fluid chamber for delivering a reagent to the particle retention region.
30. The microfluidic device according to aspect 29, wherein the microfluidic device comprises a fifth reservoir which is in fluid communication with the second fluid chamber for collecting waste from the second fluid chamber.
31. The microfluidic device according to any one of aspects 27 to 30, wherein the microfluidic device comprises a microfluidic flow system for controllably moving said particle within the second fluid chamber by fluidic force in addition to said DEP force.
32. A method of using the microfluidic device according to any one of aspects 1 to 31 and 59 to 61, the method comprising:
(a) introducing the sample comprising both the target particles and the non-target particles into the first fluid chamber,
(b) separating the target particles from the non-target particles in the first fluid chamber,
(c) flowing the target particles from the first fluid chamber to the second fluid chamber, and
(d) controlling the location of at least one of the target particles in the particle retention region in the second fluid chamber to maintain said target particle in a desired location.
33. The method according to aspect 32, wherein the separating step comprises sorting the target particles from the non-target particles based on their size difference.
34. The method according to any one of aspects 32 to 33, wherein the non-target particles are caused to pass through side openings of the first fluid chamber, and the target particles are caused to flow through the first fluid chamber and then into the second fluid chamber.
35. The method according to any one of aspects 32 to 34, wherein the target particles are cancer cells, and the non-target particles are non-cancer cells.
36. The method according to aspect 35, wherein the target particles are cancer cells such as circulating tumor cells (CTC), and the non-target particle are non-cancerous blood cells.
37. The method according to any one of aspects 32 to 36, wherein the target particles are live cells.
38. The method according to aspect 37, wherein the live cells are free of labels.
39. The method according to aspect 38, wherein the live cells are free of antibody labels.
40. The method according to any one of aspects 32 to 39, further comprising exposing at least one reagent to said target particle in the particle retention region.
41. The method according to aspect 40, wherein said target particle in the particle retention region is a cancer cell, the method further comprising measuring one or more physical, chemical and/or biological characteristics of the cancer cell in the particle retention region after exposure of the cancer cell to the at least one reagent, and the method further comprising measuring the region surrounding the cell for background correction purpose.
42. The method according to any one of aspects 40 to 41, wherein the at least one reagent comprises a chemotherapeutic drug.
43. The method according to any one of aspects 40 to 41, wherein the at least one reagent comprises a chemotherapeutic drug and a multidrug resistance (MDR) inhibitor.
44. The method according to any one of aspects 32 to 42, wherein the sample is a blood specimen of a cancer patient, and the target particle in the particle retention region is a circulating tumor cell (CTC).
45. The method according to aspect 44, further comprising generating personalized chemotherapy options for the cancer patient based on the measurements of the circulating tumor cell (CTC) in the microfluidic device.
46. A method for cell isolation and cell measurement, the method comprising:
(a) introducing a sample comprising a mixture of target cells and non-target cells into a first fluid chamber in a microfluidic device,
(b) separating the target cells from the non-target cells in the first fluid chamber based on size difference or some other physical, chemical or biological difference between the target cells and the non-target cells,
(c) flowing the target cells from the first fluid chamber to a second fluid chamber in the microfluidic device, and
(d) controlling the location of at least one of the target cells in a particle retention region in the second fluid chamber to maintain said target cell in a desired location.
47. The method according to aspect 46, further comprising:
exposing at least one reagent to said target cell in the particle retention region, and measuring one or more physical, chemical and/or biological characteristics of said cell in the particle retention region after exposure of the target cell to the at least one reagent.
48. The method according to aspect 47, wherein said at least one of the target cells is a cancer cell.
49. The method according to aspect 48, wherein said cancer cell is a circulating tumor cell (CTC).
50. The method according to any one of aspects 46 to 49, wherein the target cells are cancer cells, and the non-target cells are non-cancer cells.
51. The method according to aspect 50, wherein the target cells are cancer cells such as circulating tumor cells (CTC), and the non-target particle are non-cancerous blood cells.
52. The method according to any one of aspects 46 to 51, wherein the target cells are live cells.
53. The method according to aspect 52, wherein the live cells are free of labels.
54. The method according to aspect 53, wherein the live cells are free of antibody labels.
55. The method according to any one of aspects 47 to 54, wherein the at least one reagent comprises a chemotherapeutic drug.
56. The method according to any one of aspects 47 to 54, wherein the at least one reagent comprises a chemotherapeutic drug and a multidrug resistance (MDR) inhibitor.
57. The method according to any one of aspects 46 to 56, wherein the sample is a blood specimen of a cancer patient, and the target cell in the particle retention region is a circulating tumor cell (CTC).
58. The method according to aspect 57, further comprising generating personalized chemotherapy options for the cancer patient based on the measurements of the circulating tumor cell (CTC) in the microfluidic device.
59. The microfluidic device according to any one of aspects 1 to 31, wherein the second fluid chamber comprises a plurality of particle retention regions, each particle retention region for retaining at least one particle therein.
60. The microfluidic device according to aspect 59, wherein the second fluid chamber comprises a plurality of electrodes extending into each of said particle retention regions.
61. The microfluidic device according to any one of aspects 1 to 31, 59 and 60, wherein the microfluidic device is made of glass, quartz, plastic, polymer or some other suitable transparent material.

### REFERENCES

1. T. R. A. Ashworth, Med J Aust, 1869, 14, 146-149.
2. N. J. Caixeiro, N. Kienzle, S. H. Lim, K. J. Spring, A. Tognela, K. F. Scott, P. de Souza, and T. M. Becker, Cancer and Metastasis Reviews, 2014, 33 (2-3), 747-57.
3. K. M. Y. Mi-Young, T. Oskarsson, S. Acharyya, V. X. Nguyen, X. E. Zhang, L. Norton, and J. Massague, Cell, 2009, 139, 1315-1326
4. S. Maheswaran, D. Haber, Curr Opin Genet Dev, 2010, 20 (1), 96-99
5. C. Alix-Panabi'eres, H. Schwarzenbach, and K. Pantel, (2012) Annu. Rev. Med, 2012, 63,199-215.
6. A. Gradilone, G. Naso, C. Raimondi, E. Cortesi, O. Gandini, B. Vincenzi, R. Saltarelli, E. Chiapparino, F. Spremberg, M. Cristofanilli, L. Frati, A. M. Agliano, and P. Gazzaniga, Ann Oncol, 2011, 22 (1), 86-92.
7. F. J. Sharom, Pharmacogenomics, 2008, 9 (1), 105-127
8. B. Marquez, and F. Van Bambek, Drug Targets, 2011, 12, 600-620
9. P. R. Massay, T. Fojo, S. E. Bates, Handbook of Anticancer Pharmacokinetics and Pharmacodynamics Cancer Drug Discovery and Development, Springer, 2014, 373-400.
10. C. P. Wu, C. H. Hsieh, and Y. S. Wu, Pharmaceutics, 2011, 8 (6), 1996-2011.
11. S. Nagrath, L. V. Sequist, S. Maheswaran, D. W. Bell, D. Irimia, L. Ulkus, M. R. Smith, E. L. Kwak, D. A. Haber, and M. Toner, Nature, 2007, 450, 1235-1239
12. M. Yu, S. Stott, M. Toner, S. Maheswaran, and D. A. Haber, J. Cell. Biol, 2011, 192, 373-382.
13. M. C. Miller, G. V. Doyle, and L. W. M. Terstappen, J. Oncol, 2010, 26, 17421.
14. J. W. Allard, J. Matera Jeri, C. M. Miller, M. Repollet, M. C. Connelly, C. Rao, A. G. J. Tibbe, J. W. Uhr, and L. W.Terstappen, Clin Cancer Res, 2004, 10, 6897-6904.
15. H. M. Hou, M. Ebrahimi, B. L. Khoo, Z. R. Li, R. A. Soo, S. T. Daniel, W. T. Lim, J. Han, A. A. Bhagat, and C. T. Lim, Scientific Reports, 2013, 3, 1259-67.
16. FDA. CellSearch (TM) Circulating Tumor Cell Kit. Premarket notification (2008)
17. D. F. Hayes, M. Cristofanilli, T. G. Budd, M. J. Ellis, A. Stopeck, C. M. Miller, J. Matera, J. W. Allard, G. V. Doyle, and L. W. M. Terstappen, Clin. Cancer Res, 2006, 12, 4218-4224.
18. D. C. Danila, G. Heller, G. A. Gignac, R. Gonzalez-Espinoza, A. Anand, E. Tanaka, H. Lilja, L. Schwartz, S. Larson, M. Fleisher, and H. I Scher, Clin. Cancer Res, 2007, 13, 7053-7058.
19. S. J. Cohen, C. J. A. Punt, N. Iannotti, B. H. Saidman, K. D. Sabbath, N. Y. Gabrail, J. Picus, M. Morse, E. Mitchell, C. M. Miller, G. V. Doyle, H. Tissing, L. W. M.Terstappen, and N. J. Meropol, J. Clin. Oncol, 2008, 26, 3213-3221.
20. D. R. Shaffer, M. A. Leversha, D. C. Danila, O. Lin, R. Gonzalez-Espinoza, B. Gu, A. Anand, K. Smith, P. Maslak, G. V. Doyle, L. W. M. Terstappen, H. Lilja, G. Heller, M. Fleisher, and H. I. Scher, Clin. Cancer Res, 2007, 13, 2023-2029.
21. B. Willipinski-Stapelfeldt, S. Riethdorf, V. Assmann, U. Woelfle, T. Rau, G. Sauter, J. Heukeshoven, and K. Pantel, Clin. Cancer Res, 2005, 11, 8006-8014
22. K. Pachmann, O. Camara, A. Kavallaris, S. Krauspe, N. Malarski, M. Gajda, T. Kroll, C. Jörke, U. Hammer, A. Altendorf-Hofmann, C. Rabenstein, U. Pachmann, I. Runnebaum, and K. J. Höffken, Clin. Oncol, 2008, 26 (8), 1208-1215.
23. A. E. Ring, L. Zabaglo, M. G. Ormerod, I. E. Smith, M. Dowsett, Br J Cancer, 2005, 92 (5), 906-912.
24. I. Cruz, J. Ciudad, J. J. Cruz, M. Ramos, A. Gómez-Alonso, J. C. Adansa, C. Rodriguez, and A. Orfao, American Journal of Clinical Pathology, 2005, 123 (1), 66-74.
25. L. Zabaglo, M. G. Ormerod, M. Parton, A. Ring, I. E. Smith, and M. Dowsett, Cytometry Part A, 2003, 55A (2), 102-108.
26. H. Esmaeilsabzali, T. V. Beischlag, M. E. Cox, A. M. Parameswaran, and E. J. Park, Biotechnology advances, 2013, 31 (7), 1063-84.
27. J. Chen, J. Li, and Y. Sun, Lab Chip, 2012, 12 (10), 1753-67.
28. E. D. Pratt, C. Huang, B. G. Hawkins, J. P. Gleghorn and B. J. Kirby, Chem. Eng. Sci, 2011, 66, 1508-1522.
29. T. F. Didar and M. Tabrizian, Lab Chip, 2010, 10 (22), 3043-3053.
30. J. W. Kamande, M. L. Hupert, M. A. Witek, H. Wang, R. J. Torphy, U. Dharmasiri, S. K. Njoroge, J. M. Jackson, R. D. Aufforth, A. Snavely, J. J. Yeh, and S. A. Soper, Anal Chem, 2013, 85 (19), 9092-9100.
31. S. L. Stott, C. H. Hsu, D. I. Tsukrov, M. Yu, D. T. Miyamoto, B. A. Waltman, S. M. Rothenberg, A. M. Shah, M. E. Smas, G. K. Korir, F. P. Floyd, A. J. GilmanJ. B. Lord, D. Winokur, S. Springer, D. Irimia, S. Nagrath, L. V. Sequist, R. J. Lee, K. J. Isselbacher, S. Maheswaran, D. A. Haber, and M. Toner, Proceedings of the National Academy of Sciences, 2010, 107 (43), 18392-18397.
32. S. Nagrath, L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, M. Toner, Nature, 2007, 450 (7173), 1235-1239.
33. C. E. Earhart, R. S. Hughes, C. C. Gaster, R. J. Ooi, L. Y. Wilson, E. W. Zhou, L. Xu, D. J. Wong, and S. B. Willingham, Lab Chip, 2013, 14, 78-88.
34. K. Hoshino, Y. Y. Huang, N. Lane, M. Huebschman, J. W. Uhr, E. P. Frenkel, and X. Zhang, Lab Chip, 2011, 11 (20), 3449-3457.
35. N. Xia, T. P. Hunt, B. T. Mayers, E. Alsberg, G. M. Whitesides, R. M. Westervelt, and D. E. Ingber, Microdevices, 2006, 8 (4), 299-308.
36. K. Polyak, and R. A. Weinberg, Rev. Cancer, 2009, 9, 265-73.
37. S. B. Huang, M. H. Wu, Y. H. Lin, C. H. Hsieh, C. L. Yang, H. C. Lin, C. P. Tseng, and G. B. Lee, Lab Chip, 2013, 13 (7), 1371-1383.
38. H. S. Moon, K. Kwon, S. I. Kim, H. Han, J. Sohn, S. Lee, and H. I. Jung, Lab Chip, 2011, 11(6), 1118-1125.
39. M. D. Vahey, and J. Voldman, Anal Chem, 2008, 81 (7), 2446-2455.
40. S. B. Huang, M. H. Wu, Y. H. Lin, C. H. Hsieh, C. L. Yang, H. C. Lin, C. P. Tseng, and G. B. Lee, Lab Chip, 2013, 13 (7), 1371-1383.
41. T. M. Morgan, P. H. Lange, and R. L. Vessella, Frontiers in Bioscience, 2007, 12 (1), 3000-3009.
42. E. Sollier, E. Derek, D. E. Go, J. Che, D. R. Gossett, S. O'Byrne, W. M. Weaver, N. Kummer, M. Rettig, J. Goldman, N. Nickols, S. McCloskey, R. P. Kulkarni, and D. Di Carlo, Lab Chip, 2014, 14 (1), 63-77.
43. K. A. Hyuna, K. Kwona, H. Hanb, S. Kimb, H. Jung, Biosensors and Bioelectronics, 2103, 40 (1), 206-212.
44. R. A. Harouaka, M. Nisic, and S. Y. Zheng, S. Y. Journal of Laboratory Automation, 2013, 18 (6), 455-468.
45. A. Khamenehfar, C. P. L. Wan, P. C. H. Li, K. Letchford, and H. M. Burt, Anal. Bioanal. Chem, 2014, 406, 7071-7083.
46. X. Li, Y. Chen, and P. C. H. Li, Lab Chip, 2011, 11 (7), 1378-1384.
47. X. Li, V. Ling, and P. C. H. Li, Anal. Chem, 2008, 80 (11), 4095-102.
48. X. Li, J. Huang, G. F. Tibbits, and P. C. H. Li, Electrophoresis, 2007, 28 (24), 4723-4733.
49. J. Ji, V. Samper, Y. Chen, C. Heng, T. Lim, and L. Yobas, Biomed Microdevices, 2008, 10, 251-257.
50. Y. Zhang, J. P. Bressler, J. Neal, B. Lal, H. C. Bhang, J. Laterra, and M. G Pomper, Cancer Research, 2007, 67 (19), 9389-9397.
51. T. Liu, F. Xu, X. Du, D. Lai, Y. Zhao, Q. Huang, L. Jiang, W. Huang, W. Cheng, and Z. Liu, Mol Cell Biochem, 2010, 340, 265-273.
52. H. A. Pohl, J. Appl. Phys, 1951, 22, 869.
53. G. Bhangal, S. Halford, J. Wang, R. Roylance, R. Shah, and J. Waxman, Urol Oncol., 2000, 5 (3), 118-121
54. S. Homma, Y. Komohara, M. Harada, H. Saya, S. Todo, K. Itoh, and M. Noguchi, Oncol Rep., 2007, 18 (2), 343-346.
55. T. M. Sissung, C. E. Baum, J. Deeken, D. K. Price, J. Aragon-Ching, S. M. Steinberg, W. Dahut, A. Sparreboom, and W. D. Figg, Clin Cancer Res, 2008, 14 (14), 4543-49.
56. J. Voldman, Annu. Rev. Biomed. Eng, 2006, 8, 425-454.
57. A. Khamenehfar, Y. Chen, D. E. Hogge, P. C. H. Li, MicroTAS, 2013, 1, 380-382.
58. X. Y. Peng, and P. C. H. Li, Anal. Chem, 2004, 76 (18), 5273-5281.
59. X. Y. Peng, and P. C. H. Li, Lab Chip, 2005, 5 (11), 1298-1302.
60. Y. Zhang, M. Pullambhatla, J. Laterra, and M. G. Pomper, Molecular Imaging, 2012, 11 (6): 499-506.
61. F. Shen, X. J. Li, and P. C. H. Li, Biomicrofluidics, 2014, 8, 1-12.
62. K. Kawai, M. Sakurai, T. Sakai, M. Misaki, I. Kusano, T. Shiraishi, and R. Yatani, Cancer Lett, 2000, 150, 147-153.
63. W. J. Huss, D. R. Gray, N. M. Greenberg, J. L. Mohler, and J. L. Smith, Cancer Res, 2005, 65 (15), 6640-52.
64. W. D. Stein, Physiol Rev, 1997, 77 (2), 545-590.
65. R. Collander, Physiol. Plant, 1954, 7, 420-445.
66. N. J. Millenbaugh, M. G. Wientjes, and J. L. Au, Cancer Chemother Pharmacol, 2000, 45, 265-272.
67. Z. Fülöpa, R. Grefb, and T. Loftsson, Int. J. Pharma, 2013, 454, 559-561.
68. S. Eksborg, S. J. Pharm. Sci, 1978, 67 (6), 782-785.
69. P. O. Anderson, J. E. Knoben, and W. G. Troutman, W. G. Handbook of clinical drug data. McGraw-Hill Medical Publishing Division, 2002, 237-253.

70.F. Klepsch, I. Jabeen, P. Chiba, G. F. Ecker, Current Pharmaceutical Design, 2010, 16 (15): 1742-1752.
71. E. Lainey, N. Wolfromm, D. Enot, M. Scoazec, C. Bouteloup, C. Leroy, J. B. Micol, S. Botton, L. Galluzz, P. Fenaux, and G. Kroemer, Oncogene, 2012, 32, 4331-4342.
   All references cited herein are hereby incorporated by reference. Additionally, U.S. Pat. No. 8,124,032 entitled “Microfluidic device and method of using same” and US Patent Application Publication No. US 2012/0058504 entitled “Methods and apparatus for dielectrophoretic shuttling and measurement of single cells or other particles in microfluidic chips” are hereby incorporated by reference. For example, the second chamber (Chamber 2) of the microfluidic device according to some embodiments of the present application may comprise a fluid chamber as described and/or illustrated in U.S. Pat. No. 8,124,032 and US Patent Application Publication No. US 2012/0058504. For example, the second chamber (chamber 2) of the microfluidic device according to some embodiments of the present application may comprise a plurality of particle retention regions, each particle retention region for retaining at least one particle therein, and a plurality of electrodes extending into each of said particle retention regions for applying a DEP force to said at least one particle therein. An example chamber having a plurality of particle retention regions is described in US 2012/0058504 and illustrated in FIGS. 24A, 24B, 24C and 25 of US 2012/0058504.

1. A microfluidic device comprising:
(a) a first fluid chamber for separating target particles from non-target particles in a sample that comprises both the target particles and the non-target particles,
(b) a second fluid chamber which comprises a particle retention region for retaining at least one of the target particles, and
wherein the first fluid chamber is in fluid communication with the second fluid chamber.

2. The microfluidic device according to claim 1, wherein the first fluid chamber comprises particle selection means.

3. The microfluidic device according to claim 2, wherein the particle selection means comprises side openings along at least a side of the first fluid chamber.

4. The microfluidic device according to claim 3, wherein the side openings of the first fluid chamber have a size that allows the non-target particles to pass through, but does not allow the target particles to pass through.

5. The microfluidic device according to claim 2, wherein the first fluid chamber is an elongate fluid chamber having a first end and a second end.

6. The microfluidic device according to claim 5, wherein the microfluidic device comprises a first reservoir which is in fluid communication with the first end of the first fluid chamber, the first reservoir serving as a sample inlet reservoir.

7. The microfluidic device according to claim 5, wherein the microfluidic device comprises a second reservoir which is in fluid communication with the side openings of the first fluid chamber, the second reservoir serving as a non-target particle collection reservoir.

8. The microfluidic device according to claim 5, wherein when the sample flows in a direction from the first end of the first fluid chamber to the second end of the first fluid chamber, the non-target particles move toward the side openings whereas the target particles move in a middle portion of the first fluid chamber toward the second end.

9. The microfluidic device according to claim 8, wherein the first fluid chamber separates the target particles from the non-target particles based on size difference between the target particles and the non-target particles.

10. The microfluidic device according to claim 9, wherein the target particles are cancer cells.

11. The microfluidic device according to claim 9, wherein the non-target particles comprise non-cancer cells such as red blood cells or white blood cells or both.

12. The microfluidic device according to claim 9, wherein the target particles have a size greater than 15 μm.

13. The microfluidic device according to claim 1, wherein the second fluid chamber comprises a plurality of electrodes extending into the particle retention region for applying a dielectrophoretic (DEP) force to said at least one of the target particles to control the location of said particle within said particle retention region.

14. The microfluidic device according to claim 13, wherein the microfluidic device comprises a third reservoir which is in fluid communication with a channel connecting the first fluid chamber and the second fluid chamber, for moving one or more target particles from the first fluid chamber to the second fluid chamber.

15. The microfluidic device according to claim 14, wherein the microfluidic device comprises a fourth reservoir which is in fluid communication with the second fluid chamber for delivering a reagent to the particle retention region.

16. A method of using the microfluidic device according to claim 1, the method comprising:
(a) introducing the sample comprising both the target particles and the non-target particles into the first fluid chamber,
(b) separating the target particles from the non-target particles in the first fluid chamber,
(c) flowing the target particles from the first fluid chamber to the second fluid chamber, and
(d) controlling the location of at least one of the target particles in the particle retention region in the second fluid chamber to maintain said target particle in a desired location.

17. The method according to claim 16, wherein the non-target particles are caused to pass through side openings of the first fluid chamber, and the target particles are caused to flow through the first fluid chamber and then into the second fluid chamber.

18. The method according to claim 16, further comprising exposing at least one reagent to said target particle in the particle retention region.

19. The method according to claim 18, wherein said target particle in the particle retention region is a cancer cell, the method further comprising measuring one or more physical, chemical and/or biological characteristics of the cancer cell in the particle retention region after exposure of the cancer cell to the at least one reagent, and the method further comprising measuring the region surrounding the cell for background correction purpose.

20. The method according to claim 18, wherein the at least one reagent comprises a chemotherapeutic drug.
